(19) United States
(12) Patent Application Publication
(10) Pub. No.: US 2026/0070941 A1
(43) Pub. Date: Mar. 12, 2026

(54) Clearance of Aggregates from UF/DF Pools in Downstream Antibody Purification

(71) Applicant: AMGEN INC., Thousand Oaks, CA (US)

(72) Inventors: Ammar Arsiwala, Thousand Oaks, CA (US); Harrison Beamer, Thousand Oaks, CA (US); Dhruvkumar Upadhyay, Thousand Oaks, CA (US)

(21) Appl. No.: 18/872,079

(22) PCT Filed: Jun. 16, 2023
(86) PCT No.: PCT/US2023/025525

§ 371 Date: Dec. 5, 2024

Related U.S. Application Data
(60) Provisional application No. 63/353,776, filed on Jun. 20, 2022.

(51) Int. Cl. C07K 1/18; C07K 1/16; C07K 1/34

(52) U.S. Cl. CPC C07K 1/18; C07K 1/165; C07K 1/34

(57) ABSTRACT

A method for improving the harvest or purification of a target protein such as a biologic or biosimilar is provided. The method improves conventional harvest/purification methodologies by adding a chromatography step, such as a mixed-mode or ion exchange chromatography step, towards the end of the polishing phase of harvest/purification, after conventional chromatographic polishing steps such as protein A or ion exchange chromatography steps have been completed and the resultant eluate subjected to filtration, such as ultrafiltration/diafiltration. The surprising result of returning to chromatographic polishing after filtration is that all forms of high molecular weight products are reduced, facilitating the purification of target protein sufficient to meet government regulations, such as Quality Target Protein Profiles.

This application is a national stage application under 35 U.S.C. § 371 of International Application No. PCT/US2023/025525 filed Jun. 16, 2023, which claims priority to U.S. Provisional Application No. 63/353,776 filed Jun. 20, 2022, both of which are herein incorporated by reference in their entirety.

### FIELD

The disclosure relates generally to the field of protein harvest/purification and more specifically to the field of harvest/purification of biologic and biosimilar protein products of therapeutic value.

### BACKGROUND

Continued development of biologics and biosimilars has revealed the great promise of these molecules to provide therapeutic benefits to mankind. These molecules are large proteins that are typically produced using cell-based expression of the molecules in culture. As proteins, these molecules also often require post-translational modifications and proper folding to maximize functionality. Such considerations frequently lead to the use of eukaryotic host cells to express the desired product, although recent advances in prokaryotic expression systems signal the continuing viability of this avenue for protein expression. In addition, the development of cell culture technology has led to significant increases in the cell density of cultures producing proteins such as biologics and biosimilars, which shows promise in increasing the yield of such products but which comes at the cost of increased levels of contaminants from the cell culturing operation. In particular, the use of labile eukaryotic cells is associated with some level of cell lysis resulting in the contaminating presence of unwanted biomolecules such as nucleic acids, proteins, lipids and the like along with cell membrane fragments. Given the potential gains in yield from use of high density cell cultures, a need exists for harvest or purification methods that address the increased presence of contaminants.

Traditional purification protocols for proteins expressed in cell culture involve upstream cell culture effluent processing focused on various forms of centrifugation and/or various forms of depth filtration, with an optional initial step of flocculation or precipitation to remove some of the contaminants in the effluent. Downstream polishing steps have involved one or more chromatographic fractionations involving, for example affinity chromatography, ion exchange chromatography, hydrophobic interaction chromatography, and other forms of chromatography. A typical affinity chromatography step involved in polishing the purification of target proteins such as biologics and biosimilars has been protein A chromatography, which is well-known for use in purifying immunoglobulin-like proteins such as antibodies. Ion exchange chromatography often involves anion exchange but can also involve cation exchange. Following the train of one or more chromatographic fractionations in the downstream polishing steps of a harvest/purification methodology, there is often a filtration step such as an ultrafiltration step, which may involve diafiltration, a form of ultrafiltration with solvent replenishment. Following filtration, the purified protein is typically formulated for use such as administration to subjects in need.

The use of cell-culture-based expression systems to produce therapeutic biologics and biosimilars has required these multi-step purification protocols to ensure sufficient purity of products destined for administration to humans. To ensure sufficient purity, governmental regulators throughout the world have developed stringent standards that must be met, including the requirement in the US to meet Quality Target Protein Profiles, which are rigorous standards for the purification of products to be administered to humans. The requirement to obtain governmental approval to administer biologics and biosimilars has resulted in continuing demand for greater purity of products expressed in culture. Thus, a need continues to exist in the art for improvements to protein harvest/purification methodologies to yield protein target products with reduced levels and types of culture-based contaminants.

### SUMMARY

The disclosure provides methodologies that improve the purity of target proteins, such as biologics and biosimilars, that are produced in cell culture. The disclosure takes an approach at odds with conventional technologies in the downstream aspect of protein purification from cell culture. More particularly, in the downstream polishing phase of protein purification from cell culture, the disclosure relies on one or more chromatography steps prior to an ultrafiltration/diafiltration (UF/DF) step, consistent with conventional purification protocols, but then takes the unusual step of returning to chromatographic fractionation involving at least a mixed-mode chromatography or an ion exchange chromatography (e.g., cation exchange chromatography) step. The purification methodology of the disclosure results in a significant reduction of undesired high molecular weight (HMW) material contaminating the purified target protein material while minimizing reductions in yield of that target protein. In adding this unusual step late in the polishing phase of target harvest/purification, the method provides improved removal of all forms of high molecular weight compounds, including nucleic acids, proteins, lipids, and other forms of high molecular weight compounds found in cell-based target protein production methods. The result is a target protein of greater purity that exhibits an improved profile conforming to Quality Target Protein Profiles.

In one aspect, the disclosure provides a method of harvesting a target protein from a host cell culture fluid comprising a chromatography step after an ultrafiltration/diafiltration (UF/DF) step, wherein the high molecular weight species in the eluate from the chromatography step are reduced by at least 10% compared to the level of high molecular weight species in the UF/DF filtrate. In some embodiments, the method further comprises at least one chromatography step preceding the ultrafiltration/diafiltration step. In some embodiments, the at least one chromatography step comprises protein A chromatography. In some embodiments, the at least one chromatography step further comprises ion exchange chromatography, mixed-mode chromatography, or both ion exchange chromatography and mixed-mode chromatography. In some embodiments, the method further comprises upstream bulk harvest steps of centrifuging, depth filtering, or both centrifuging and depth filtering the host cell culture fluid followed by downstream polishing steps to purify the target protein, wherein the polishing steps comprise a protein A chromatography step, a low pH viral inactivation step, a cation exchange step, a mixed-mode anion exchange chromatography step, a viral filtration step, an ultrafiltration/diafiltration step, the chromatography step after the ultrafiltration/diafiltration step, a polysorbate 80 addition step, and a final filtration step.

Still other embodiments of the method are provided wherein the target protein subjected to the chromatography step is present in a formulation buffer comprising 10 mM glutamic acid, 250 mM threonine, pH 5.5. In some embodiments, the output from the chromatography step is an eluate wherein the high molecular weight compounds are reduced by at least 9% compared to the level in the UF/DF filtrate. In some embodiments, the high molecular weight compounds in the eluate are reduced by at least 20%, at least 25%, at least 30%, or at least 75% compared to the level in the UF/DF filtrate. In some embodiments, the output from the chromatography step is an eluate comprising 0.3-1.9% high molecular weight compounds. In some embodiments, the yield of target protein from the chromatography step is at least 58%, 60%, 65%, 70%, 75%, 80%, 86%, at least 90%, at least 95%, or at least 98%.

Some embodiments of the method are disclosed wherein the chromatography media is a mixed-mode resin, a mixed-mode membrane, an ion exchange resin or an ion exchange membrane. In some embodiments, the chromatography media is Ca++Pure-HA, Capto MMC, Capto MMC ImpRes, Capto SP ImpRes, Capto Adhere, CIMultus PrimaS, CIMultus Hbond, CMM Hypercel, Eshmuno CP-FT, Eshmuno HCX, Fibro MMC, Fibro Adhere, Fractogel COO— (M), Fractogel SO3- (M), Mustang XT S, Nuvia S, Nuvia HR-S, Nuvia cPrime, Sartobind Phenyl, ToyoPearl MX-Trp, ToyoPearl Sulfate 650M, or UNOsphere S. In some embodiments, the chromatography media is Ca++ Pure HA, Capto MMC, Capto MMC ImpRes, Capto Adhere, CMM Hypercel, Eshmuno HCX, Fibro MMC, Fibro Adhere, Nuvia cPrime, ToyoPearl mX-Trp, or ToyoPearl Sulfate. In some embodiments, the chromatography media is Capto MMC ImpRes, Eshmuno HCX, Eshmuno CP-FT, Fibro MMC, or Nuvia cPrime. In some embodiments, the chromatography media is Fibro MMC or Nuvia cPrime. In some embodiments, the chromatography media is a membrane. In some embodiments, the fluid comprising the target protein is applied to the chromatography media with a load factor of at least 400 grams/Liter-resin, including embodiments wherein the load factor is between 400-800 grams/Liter-resin and embodiments wherein the load factor is at least 800 grams/Liter-resin.

The method also provides embodiments wherein the load % HMW is at least 0.5%. In some embodiments, the load % HMW is at least 0.9%, is at least 1.2%, is between 0/5-2.6%, or is at least 2.6%. In some embodiments of the method, the chromatography step yields a % HMW clearance of at least 5%, at least 8%, at least 24%, at least 30%, at least 33%, at least 40%, at least 45%, at least 55%, at least 65%, at least 70%, or at least 75%.

The method also provides embodiments wherein the fluid comprising the target protein is applied to the chromatography media at a load concentration of no more than 50 g/L. In some embodiments, the load concentration is no more than 20 g/L. In some embodiments of the method, the chromatography media comprises a ligand at a ligand density of at least 98 mmol/mL chromatography media. In some embodiments, the ligand density is between 98-157 mmol/mL, is between 98-140 mmol/mL, or is between 127-157 mmol/mL. In some embodiments, the eluate from the chromatography step has a Quality Target Protein Profile of 0.3% or less, wherein the eluate comprises the target protein.

The disclosure will be better understood upon consideration of the following detailed description of the disclosure, including a consideration of the figures.

### BRIEF DESCRIPTION OF THE DRAWING

FIG. 1. Bar graphs demonstrating the cross-molecule applicability of adding a post-UF/DF chromatography step to a purification protocol for a large protein such as an antibody biologic/biosimilar. A worst-case load material level of 3.4% HMW was used in assessing the performance of the post-UF/DF chromatography step when purifying mAb4 from culture fluid. Performance was assessed by determining % HMW as a measure of HMW clearance and by determining % step yield. The tested resins were CaptoMMC ImpRes, Nuvia cPrime, CMM Hypercel, ToyoPearl Sulfate, and Capto Adhere. Nuvia cPrime resin provided 24% HMW clearance with a % step yield greater than 90%. The results demonstrate that addition of a post-UF/DF chromatography step in biologic/biosimilar purification protocols has wide applicability in showing significant reduction of HMW and desirably high step yields across multiple biologic/biosimilars being purified. The % HMW clearance—salmon-colored bars; the % step yield—gray bars; load % HMW—dotted line.

FIG. 2. Bar graphs showing the effect of load factor (grams/liter-resin (g/L-r)) on HMW clearance and % step yield when incorporating a post-UF/DF chromatography step in purifying mAb4 from culture fluid. The load material (i.e., target-containing fluid including any HMW materials (any HMW species)) contained 1.5% HMW. Load factors of 800 g/L-r (high) and 400 g/L-r (low) were assessed, as indicated in the Figure. The resins tested were Nuvia cPrime (800 g/L-r), Nuvia cPrime (400 g/L-r), ToyoPearl Sulfate (800 g/L-r) and ToyoPearl Sulfate (400 g/L-r). Nuvia cPrime (400 g/L-r) provided 33% HMW clearance. Pool % HMW—salmon-colored bars; % step yield—gray bars; load % HMW—dotted line.

### DETAILED DESCRIPTION

Data disclosed herein establishes that a post-ultrafiltration/diafiltration (post-UF/DF) chromatography step added relatively late in a purification regimen can significantly reduce the concentration of high molecular weight contaminants when purifying relatively high molecular weight target molecules such as therapeutic biologics and biosimilars. Moreover, the experimental results reveal that the post-UF/DF chromatography step can be effectively performed using resin-based or membrane-based chromatography media.

As used herein, “load factor” is the mass amount of protein in the material applied to the post-UF/DF chromatography media per liter of chromatographic media or resin. The load factor is specified in units of grams/liter-resin. “High molecular weight” or HMW refers to species that are at least 10,000 daltons. “Load % HMW” is the percentage of the overall mass of the material applied to the post-UF/DF chromatography media that is composed of high molecular weight species. “% HMW” is the percent high molecular weight species. “% HMW clearance” or “percent HMW reduction” is the percentage of HW removed during the post-UF/DF chromatography. % HMW clearance=(load % HMW−pool % HMW)/load % HMW. “Pool % HMW” is the percentage of HMW species in the eluate from the post-UF/DF chromatography. “% step yield” is the yield of protein resulting from post-UF/DF chromatography.

The following Examples disclose the experimental assessments of subjecting different monoclonal antibodies, i.e., mAb3 (IgG1), and mAb4 (IgG1), to post-UF/DF chromatography using various resins to reduce HMW while providing high yields of the proteins, such as biologics and biosimilars.

### EXAMPLES

### Example 1

### Materials

TABLE 1
List of materials used in the experiments

| Material | Description | | Catalog # |
|---|---|---|---|
| mAb3 | IgG1 | | N/A |
| mAb4 | IgG1 | | N/A |
| Eshmuno CP-FT (Millipore Sigma) | 1 | mL column | |
| Fractogel COO— (EMD/Millipore | 1 | mL column | |
| sigma) | | | |
| Fractogel SO3— (EMD/Millipore | 1 | mL column | |
| sigma) | | | |
| Nuvia HR-S (Biorad Laboratories) | 1 | mL column | |
| UNOsphere S (Biorad Laboratories) | 1 | mL column | |
| ToyoPearl MX-Trp (Tosoh | 1 | mL column | |
| Biosciences) | | | |
| Mustang XT S (PALL) | 0.86 | mL membrane | |
| Sartobind Phenyl (Sartorius Stedim | 3 | mL membrane | |
| Biotech) | | | |
| Capto MMC ImpRes (Cytiva) | 1 | mL column | |
| ToyoPearl Sulfate (Tosoh | 1 | mL column | |
| Biosciences) | | | |
| Eshmuno HCX (Millipore Sigma) | 1 | mL column | |
| Nuvia cPrime (Biorad Laboratories) | 1 | mL column | |
| Capto Adhere (Cytiva) | 1 | mL column | |
| Ca++ Pure-HA (Tosoh | 1 | mL column | |
| Biosciences) | | | |
| Capto MMC (Cytiva) | 1 | mL column | |
| Sartorius CMM Hypercel (Sartorius | 1 | mL column | |
| Stedim Biotech) | | | |
| CIMultus PrimaS (Sartorius Stedim | 1 | mL column | |
| Biotech) | | | |
| CIMultus Hbond (Sartorius Stedim | 1 | mL column | |
| Biotech) | | | |
| Nuvia S (Biorad Laboratories) | 1 | mL column | |

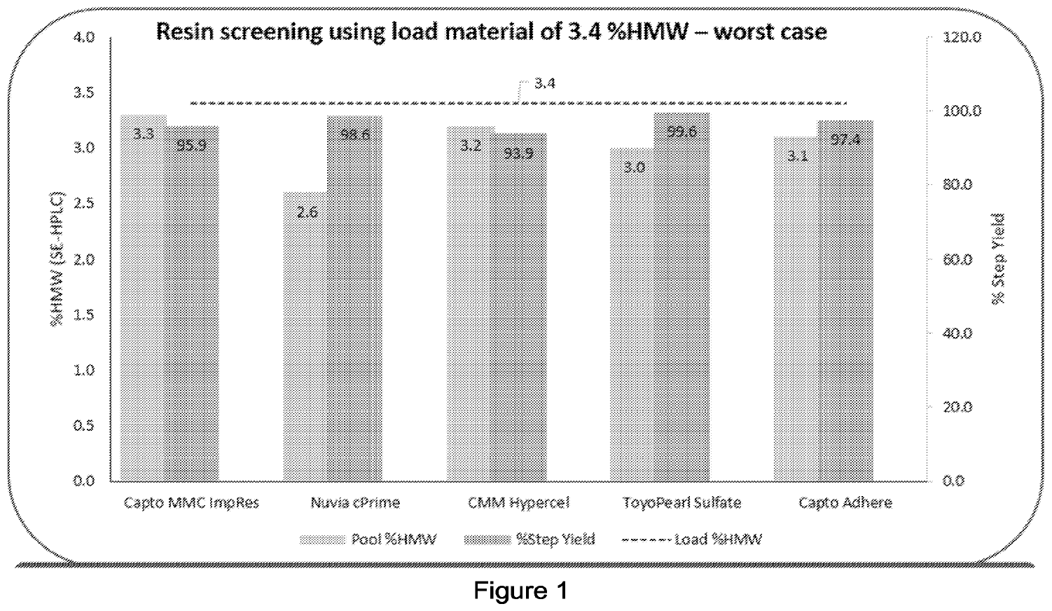

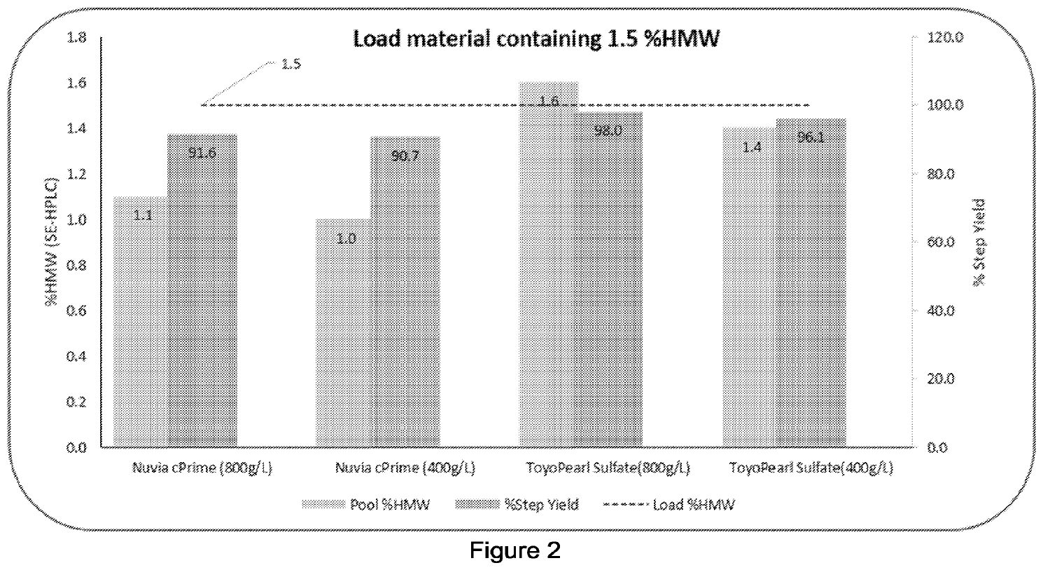

Initial screens of chromatographic media used in post-UF/DF chromatography of mAb4 fluids yielded the data shown in FIG. 2, as described in Example 2. The results established that Capto MMC ImpRes, Nuvia cPrime, CMM Hypercel, ToyoPearl Sulfate and Capto Adhere were the top chromatographic media in removing undesired HMW while providing high yields of mAb4 target protein. These top chromatographic media were subjected to a worst-case load % HMW of 3.4% in mAb4-containing fluids. The results are presented in FIG. 1 and show a reduction in % HMW while maintaining high % step yields of at least 93.9%.

The experimental results presented in the following Examples and in the Figures reflect rigorous experimental analyses of multiple chromatorgraphic modalities for use in a post-UF/DF harvest/purification step for target proteins such as monocloncal antibodies. In particular, the disclosure presents analyses of (1) cation exchange chromatography (Eshmuno CP-FT, Fractogel COO— (M), Mustang XT S, CIMultus PrimaS, Nuvia 5, Fractogel SO3— (M), Nuvia HR-S, Fibro Adhere, Fibro MMC, and UNOsphere S), (2) hydrophobic interaction chromatography (Sartobind Phenyl), and (3) mixed-modal exchange chromatography (ToyoPearl MX-Trp, Capto Adhere, Ca⁺⁺ Pure HA, Capto MMC, Eshmuno HCX, Nuvia cPrime, Capto MMC ImpRes, CMM Hypercel, and Toyopearl Sulfate 650M). The results identify multi-modal or mixed-modal chromatography media as best suited for use in this chromatographic step in terms of % HMW removal with significant yield of target protein.

### Example 2

### Monoclonal Antibody 3 (mAb3)

Monoclonal antibody 3 (mAb3) is an antibody of the IgG1 subclass with a pl of 9.0. mAb3 material was produced in a large-scale run, where it was buffer-exchanged from a matrix of 100 mM sodium acetate, 200 mM sodium chloride, pH 5.0 to the formulation buffer containing 6.4 mM L-histidine, 7.6% sucrose, pH 6.0 (see Table 2) and the pool was concentrated to a final concentration of about 88 g/L. The % HMW in the mAb3 UF/DF pool was about 0.4%.

Monoclonal antibody 3 material, produced in a large-scale run, was also buffer-exchanged into a matrix of the formulation buffer containing 10 mM sodium phosphate, 10% (w/v) sucrose, pH 7.2, concentrating the pool to a final concentration of about 19 g/L.

To generate a mAb3 UF/DF pool containing a high level of HMW (about 1%), a part of the original pool (at 0.2% HMW) was held at a low pH of 3.0 for 10 minutes at ambient temperature, neutralized to pH 6.0 using 2 M Tris Base, and then spiked back into the original pool to elevate the level of % HMW. As also mentioned in Example 3, the mAb3 UF/DF pool was stressed at 50° C. for two weeks to generate a pool at 1.7% HMW for the runs loaded at 1200 g/L-r.

The sequence of steps shown in Table 2 was followed for chromatography operations for mAb3.

TABLE 2
Sequence of steps for chromatography operations for mAb3

| | | Volume Required |
|---|---|---|
| Phase | Solution Description | (CV) |
| Equilibration | mAb3 formulation buffer | 10 |
| | (6.4 mM L-Histidine, 7.6% w/v | |
| | Sucrose, pH 6.0) | |
| Load | mAb3 UF/DF pool (@88 g/L) | As needed (400 or |
| | | 800 g/Lr) |
| | | Start collect 0.5 OD |
| | | (UV280) |
| Wash | mAb3 formulation buffer | Up to 30 CV |
| | (6.4 mM L-Histidine, 7.6% w/v | Stop collect 1.0 OD |
| | Sucrose, pH 6.0) | (UV 280) |
| Strip | 6.4 mM L-Histidine, 1M Sodium | 5 |
| | Chloride, pH 6.4 | |
| Denaturing | 1M NaOH (certain resins 0.1M | 5 |
| Cleaning | NaOH, or 0.5M NaOH,) | |
| Storage | Per resin recommendation | 3 |

### Example 3

### Initial Resin Screening

An initial resin screen was performed by loading mAb3 at about 88 g/L with a load factor of 800 g/L-r on the resins/membranes listed in Table 3. The chromatography device was a pre-packed column, with the corresponding column or membrane volume of 1 mL, consistent with the volumes listed in Table 1.

The % HMW reduction was calculated as ((% HMW[load]−% HMW[pool])/% HMW[load]) to represent the % reduction in % HMW. The protein concentration of mAb1 was measured using a CTech SoloVPE System (Repligen) (extinction coefficient of 1.54 mg/mL*cm). Table 3 summarizes the experimental findings with respect to % HMW reduction and % step yield.

TABLE 3
% HMW Clearance of initial resin screening for mAb3

| | | Load Factor | Load | Pool | % HMW | Yield |
|---|---|---|---|---|---|---|
| Resin/Membrane | Run # | (g/Lr) | % HMW | % HMW | Reduction | % |
| Capto MMC ImpRes | R01 | 800 | 0.823 | 0.664 | 19.3 | 77.43 |
| ToyoPearl Sulfate | R02 | 800 | 0.823 | 0.810 | 1.5 | 87.47 |
| Eshmuno HCX | R03 | 800 | 0.823 | 0.952 | −15.7 | 94.35 |
| Nuvia cPrime | R04 | 800 | 0.823 | 0.685 | 16.8 | 92.04 |
| Capto Adhere | R05 | 800 | 0.823 | 0.920 | −11.8 | 101.12 |
| Ca++ Pure HA | R06 | 800 | 0.823 | 0.691 | 16.0 | 92.08 |
| Eshmuno CP-FT | R07 | 800 | 0.823 | 0.530 | 35.6 | 79.15 |
| ToyoPearl MX-Trp | R08 | 800 | 0.823 | 0.588 | 28.5 | 85.30 |
| Fractogel COO— | R09 | 800 | 0.823 | 0.646 | 21.4 | 85.87 |
| Unosphere S | R10 | 800 | 0.823 | 0.871 | −5.9 | 90.26 |

### Example 4

### Effect of Load Factor

The initial screening experiments disclosed in Example 2 were performed at a high load factor of 800 g/L-r. Additionally, runs were performed at a lower load factor of 400 g/L-r, and two runs at a higher load factor of 1200 g/L-r. The mAb3 load contained about 1.0-1.8% HMW. The 1.7% Load HMW was achieved by stressing the mAb3 pool for two weeks at 50° C. for the 1200 g/L-r runs as a worst-case example.

Table 4 summarizes the findings on the effect of load factor on the HMW clearance ability and % step yield of mAb3 using Nuvia cPrime, Eshmuno CP-FT, ToyoPearl MX-Trp, and Fractogel COO—, i.e., the top four resins identified from the initial screens.

TABLE 4
% HMW Clearance and % step yield of Nuvia cPrime, Eshmuno CP-FT,
ToyoPearl MX-Trp, and Fractogel COO— at 400, 800, and 1200 g/Lr

| | | Load Factor | Load | Pool | % HMW | Yield |
|---|---|---|---|---|---|---|
| Resin/Membrane | Run # | (g/Lr) | % HMW | % HMW | Reduction | % |
| Nuvia cPrime | R15 | 400 | 1.028 | 0.922 | 10.3 | 69.20 |
| Eshmuno CP-FT | R16 | | 1.028 | 0.660 | 35.8 | 58.56 |
| ToyoPearl MX-Trp | R17 | | 1.028 | 0.702 | 31.7 | 69.42 |
| Fractogel COO— | R18 | | 1.028 | 0.996 | 3.1 | 68.10 |
| Nuvia cPrime | R11 | 800 | 1.028 | 0.852 | 17.1 | 77.51 |
| Eshmuno CP-FT | R12 | | 1.028 | 0.684 | 33.5 | 75.49 |
| ToyoPearl MX-Trp | R13 | | 1.028 | 0.753 | 26.7 | 77.09 |
| Fractogel COO— | R14 | | 1.028 | 0.857 | 16.6 | 77.95 |
| Eshmuno CP-FT | R19 | 1200 | 1.752 | 1.243 | 29.1 | 69.51 |
| Fractogel COO— | R21 | | 1.752 | 1.469 | 16.2 | 84.63 |

### Example 5

### Monoclonal Antibody 4 (mAb4)

To generate a monoclonal antibody 4 (mAb4) post-UF/DF pool containing a high level of HMW (about 3%), a part of the original pool was held at a low pH of 3.4 for up to 7 hours at 30° C., then neutralized to pH 7.2 using 2 M Tris Base. Each section of the experiment details the duration of the high-temperature stress. The stressed material was loaded onto the columns without any spiking.

Table 5 shows the sequence of steps that were executed for each of the mAb4 experiments.

TABLE 5
Sequence of steps for chromatography operations for mAb4

| | | Volume Required |
|---|---|---|
| Phase | Solution Description | (CV) |
| Equilibration | mAb4 formulation buffer | 10 |
| | (10 mM sodium phosphate, 10% | |
| | (w/v) sucrose, pH 7.2) | |
| Load | mAb4 UF/DF pool (@19 g/L) | As needed (400 or |
| | | 800 g/L-r) |
| | | Start collect 0.5 OD |
| | | (UV280) |
| Wash | mAb3 formulation buffer | Up to 30 CV |
| | (10 mM sodium phosphate, 10% | Stop collect 1.0 OD |
| | (w/v) sucrose pH 7.2) | (UV 280) |
| Strip | 10 mM sodium phosphate, 1M | 5 |
| | sodium chloride, pH 7.2 | |
| Denaturing | 1M NaOH (certain resins 0.1M | 5 |
| Cleaning | NaOH, or 0.5M NaOH) | |
| Storage | Per resin recommendation | 3 |

### Example 6

### 5.1. Initial Resin Screen

An initial resin screen was performed by loading mAb4 at about 19 g/L with a load factor of 800 g/L-r on the resins/membranes listed in Table 5. The chromatography device was a pre-packed column, with the corresponding 1 mL column volumes listed in Table 1.

The % HMW reduction was calculated as ((% HMW[load]−% HMW[pool])/% HMW[load]) to represent the % reduction in % HMW. The protein concentration of mAb4 was measured using a CTech SoloVPE System (Repligen) (extinction coefficient of 1.40 mg/mL*cm). Table 6 summarizes the experimental findings with respect to % HMW reduction and % step yield.

TABLE 6
% HMW Clearance and % step yield of the initial resins screened for mAb4

| | | Load Factor | Load | Pool | % HMW | Yield |
|---|---|---|---|---|---|---|
| Resin/Membrane | Run # | (g/Lr) | % HMW | % HMW | Reduction | % |
| Capto MMC ImpRes | R01 | 800 | 3.353 | 3.307 | 1.4 | 95.89 |
| Nuvia cPrime | R02 | 800 | 3.353 | 2.621 | 21.8 | 98.63 |
| Sartorius CMM Hypercel | R03 | 800 | 3.353 | 3.156 | 5.9 | 93.87 |
| ToyoPearl Sulfate | R04 | 800 | 3.353 | 3.041 | 9.3 | 99.63 |
| Capto Adhere | R05 | 800 | 3.353 | 3.1 | 7.5 | 97.40 |

### Example 7

### Effect of Load Factor

The initial screening experiments discussed in Example 6 were performed at a high load factor of 800 g/L-r. Additional runs were performed at a lower load factor of 400 g/L-r for the two resins with the highest % HMW reduction (Nuvia cPrime and ToyoPearl Sulfate). The mAb4 load was stressed at pH 3.4 for two hours at 30° C. and then neutralized to pH 7.2. This load contained 1.5% HMW.

Table 7 summarizes the effect of load factor on HMW clearance ability and % step yield for Nuvia cPrime and ToyoPearl Sulfate, i.e., the top two resins identified in the initial screens (see Example 6 and Table 6) at both 800 g/L-r and 400 g/L-r.

TABLE 7
% HMW and % step yield of Nuvia cPrime and
ToyoPearl Sulfate loaded at 800 & 400 g/L-r

| | | Load Factor | Load | Pool | % HMW | Yield |
|---|---|---|---|---|---|---|
| Resin/Membrane | Run # | (g/Lr) | % HMW | % HMW | Reduction | % |
| Nuvia cPrime | R06 | 800 | 1.537 | 1.137 | 26.0 | 91.55 |
| ToyoPearl Sulfate | R08 | | 1.537 | 1.564 | −1.8 | 97.97 |
| Nuvia cPrime | R07 | 400 | 1.537 | 0.962 | 37.4 | 90.75 |
| ToyoPearl Sulfate | R09 | | 1.537 | 1.416 | 7.9 | 96.12 |

All references cited throughout the application are incorporated herein by reference in their entireties or in pertinent part, as would be apparent from context. The disclosure has presented embodiments to facilitate disclosure of the subject matter, but the only limitations to be placed upon the disclosure are the limitations found in the claims.

1. A method of harvesting a target protein from a host cell culture fluid comprising a chromatography step after an ultrafiltration/diafiltration (UF/DF) step, wherein the high molecular weight species in the eluate from the chromatography step are reduced by at least 10% compared to the level of high molecular weight species in the UF/DF filtrate.

2. The method of claim 1 further comprising at least one chromatography step preceding the ultrafiltration/diafiltration step.

3. The method of claim 2 wherein the at least one chromatography step preceding the ultrafiltration/diafiltration step comprises protein A chromatography.

4. The method of claim 3 wherein the at least one chromatography step preceding the ultrafiltration/diafiltration step further comprises ion exchange chromatography, mixed-mode chromatography, or both ion exchange chromatography and mixed-mode chromatography.

5. The method of claim 1 further comprising upstream bulk harvest steps of centrifuging, depth filtering, or both centrifuging and depth filtering the host cell culture fluid followed by downstream polishing steps to purify the target protein, wherein the polishing steps comprise a protein A chromatography step, a low pH viral inactivation step, a cation exchange step, a mixed-mode anion exchange chromatography step, a viral filtration step, an ultrafiltration/diafiltration step, the chromatography step after the ultrafiltration/diafiltration step, a polysorbate 80 addition step, and a final filtration step.

6-7. (canceled)

8. The method of claim 1 wherein the high molecular weight species in the eluate are reduced by at least 20% compared to the level in the UF/DF filtrate.

9. (canceled)

10. The method of claim 8 wherein the high molecular weight species in the eluate are reduced by at least 30% compared to the level in the UF/DF filtrate.

11. The method of claim 8 wherein the high molecular weight species in the eluate are reduced by 75% compared to the level in the UF/DF filtrate.

12. The method of claim 1 wherein the output from the chromatography step is an eluate comprising 0.3-1.9% high molecular weight species.

13. The method of claim 1 wherein the yield of target protein from the chromatography step is at least 58%.

14. The method of claim 13 wherein the yield of target protein from the chromatography step is at least 86%.

15. The method of claim 13 wherein the yield of target protein from the chromatography step is at least 90%.

16-17. (canceled)

18. The method of claim 1 wherein the chromatography step after the ultrafiltration/diafiltration step comprises using a chromatography media, wherein the chromatography media is a mixed-mode resin, a mixed-mode membrane, an ion exchange resin or an ion exchange membrane.

19. The method of claim 18 wherein the chromatography media is Ca++Pure-HA, Capto MMC, Capto MMC lmpRes, Capto SP lmpRes, Capto Adhere, CIMultus PrimaS, CIMultus Hbond, CMM Hypercel, Eshmuno CP-FT, Eshmuno HCX, Fibro MMC, Fibro Adhere, Fractogel COO— (M), Fractogel SO3— (M), Mustang XT S, Nuvia S, Nuvia HR-S, Nuvia cPrime, Sartobind Phenyl, ToyoPearl MX-Trp, ToyoPearl Sulfate 650M, or UNOsphere S.

20. The method of claim 18 wherein the chromatography media is Ca++ Pure HA, Capto MMC, Capto MMC lmpRes, Capto Adhere, CMM Hypercel, Eshmuno HCX, Fibro MMC, Fibro Adhere, Nuvia cPrime, ToyoPearl mX-Trp, or ToyoPearl Sulfate.

21. The method of claim 20 wherein the chromatography media is Capto MMC lmpRes, Eshmuno HCX, Eshmuno GP-FT, Fibro MMC, or Nuvia cPrime.

22. The method of claim 21 wherein the chromatography media is Fibro MMC or Nuvia cPrime.

23. The method of claim 19 wherein the chromatography media is a membrane.

24. (canceled)

25. The method of claim 1 wherein the load factor is between 400-800 grams/Liter-resin.

26. The method of claim 1 wherein the load factor is at least 800 grams/Liter-resin.

27. (canceled)

28. The method of claim 1 wherein the load % HMW is at least 0.9%.

29. The method of claim 1 wherein the load % HMW is at least 1.2%.

30. The method of claim 1 wherein the load % HMW is between 0.5-2.6%.

31. The method of claim 1 wherein the load % HMW is at least 2.6%.

32-42. (canceled)

43. The method of claim 1 wherein the fluid comprising the target protein is applied to the chromatography media at a load concentration of no more than 50 g/L.

44. The method of claim 43 wherein the load concentration is no more than 20 g/L.

45. The method of claim 1 wherein the chromatography media comprises a ligand at a ligand density of at least 98 mmol/ml chromatography media.

46-48. (canceled)

49. The method of claim 1 wherein the eluate from the chromatography step has a Quality Target Protein Profile of 0.3% or less, wherein the eluate comprises the target protein.

50. The method of claim 1, wherein the chromatography step after the ultrafiltration/diafiltration step comprises mixed-mode chromatography.
